(19) United States
(12) Patent Application Publication
(10) Pub. No.: US 2005/0043673 A1
(43) Pub. Date: Feb. 24, 2005

(54) Method and apparatus for irrigation and drainage of the brain's subdural space using a percutaneous approach

(76) Inventor: Daniel M. Lieberman, Phoenix, AZ (US)

Correspondence Address: WEISS & MOY PC, 4204 NORTH BROWN AVENUE, SCOTTSDALE, AZ, 85251, US

(21) Appl. No.: 10/646,903

(22) Filed: Aug. 22, 2003

(51) Int. Cl. A61M 1/00

(52) U.S. Cl. 604/28; 604/43

(57) ABSTRACT

A dual lumen catheter provides for percutaneous drainage of a subdural hematoma to irrigate and drain the subdural space. The dual lumen catheter comprises a drainage channel dimensioned to drain subdural fluid collection from the subdural space and an irrigation channel dimensioned to irrigate the subdural space. Several methods are disclosed for inserting the dual lumen catheter into the subdural space through a small burr hole drilled in the skull. The dual lumen catheter 310 then both irrigates and drains the subdural space in order to both effectively evacuate a subdural hematoma as well as cleanse the subdural space. The subdural space is thereby effectively collapsed, and the brain returns to be adjacent to the dura mater and skull.

### RELATED APPLICATION

This application claims priority to a corresponding provisional application Express Mail Label No. EU686248408US, filed Dec. 16, 2002 in the name of the applicant of this application.

### FIELD OF THE INVENTION

This invention pertains in general to devices and procedures for medical care and pertains in particular to medical devices and techniques for percutaneous drainage.

### BACKGROUND OF THE INVENTION

Subdural hematomas typically result from traumatic head injuries. An example subdural hematoma is explained with respect to FIGS. 1A and 1B.

FIG. 1A illustrates a typical subdural space. As shown in FIG. 1A, a bridging vein 110 bridges a brain 120 and dura mater 130, thereby connecting a skull 140 and the brain 120. Subdural space 125 is shown between the dura mater 130 and the brain 120.

FIG. 1B illustrates a typical subdural hematoma 150. A subdural hematoma generally results from tearing of the bridging vein 110. Subdural hematomas usually resolve spontaneously through the lyses of red cells. However, subdural hematomas can also persist as chronic fluid collections. By dilating the subdural space 125, the subdural hematoma 150 can lead to the tearing of additional, neighboring veins. The tearing of additional neighboring veins can cause further bleeding, sometimes acute bleeding, into the chronic subdural fluid collection thereby further dilating the subdural. As a result, such further bleeding can propagate the subdural hematoma 150. Subdural fluid collections can also expand dangerously even in the absence of acute hemorrhage either by absorption of fluid from the adjacent brain by osmosis or through the creation of a zone of local coagulapathy within the hematoma resulting from the release of coagulation factors. Accordingly, it is necessary to effectively treat the subdural hematoma to minimize such expansion and propagation risks.

FIGS. 1C and 1D illustrate known techniques for treating subdural hematomas. In symptomatic cases, chronic subdural hematomas are typically treated by evacuation using one of two different techniques: (1) multiple burr holes (as shown in FIG. 1C) or (2) percutaneous drainage (as shown in FIG. 1D).

Referring now to FIG. 1C, evacuation using multiple burr holes reduces pressure on the brain 120 by draining the subdural hematoma 150 through large burr holes 163 in the skull 140, followed by copious irrigation of the subdural space 125 to wash out the errant coagulation factors. This technique requires drilling large burr holes 163 through the skull 140 and is performed in an operating room. A disadvantage of this technique of using large burr holes 163 for the evacuation of chronic subdural hematomas is that rapid evacuation of the subdural hematoma 150 creates a new space adjacent the brain previously occupied by the subdural hematoma 150. If the brain 120 does not expand to fill this space then a subdural hematoma 150 can recur.

As shown in FIG. 1D, percutaneous catheter drainage has emerged as a less invasive technique for the treatment of chronic subdural hematomas, which may be performed at the patient's bedside. A catheter is inserted through a single burr hole 163 in the skull 140 into the subdural space 125 and connected to a drainage bag (not shown). Using this technique, the subdural hematoma 150 is drained slowly, over a period of days, allowing the brain 120 to gradually expand out toward the skull 140 without creation of a new space, thereby reducing the risk of subdural hematoma recurrence. However, a disadvantage of this technique, compared to the technique shown in FIG. 1C, is the inability to irrigate the subdural space 125 to effectively wash out coagulation products.

Accordingly, there is a need in the art for an improved technique to perform treatment of a subdural hematoma 150. Preferably, an improved technique will not require the drilling of multiple large burr holes 163 but will also effectively wash out coagulation products.

### SUMMARY OF THE INVENTION

An object of the present invention is to provide a dual lumen catheter capable of both irrigating and draining the brain's subdural space for percutaneous drainage of a subdural hematoma.

It is a further object of the present invention to provide a method for treating a subdural hematoma by inserting a dual lumen catheter into a subdural space in order to both drain and irrigate the subdural space evacuating the subdural hematoma.

### BRIEF DESCRIPTION OF THE PREFERRED EMBODIMENTS

In accordance with one embodiment of the present invention, an apparatus for use in medical procedures for treating subdural hematomas is disclosed, comprising a dual lumen catheter comprising, in combination, a drainage channel having a proximal portion and a distal portion, and an irrigation channel having a proximal portion and a distal portion.

In accordance with another embodiment of the present invention, a method for treating subdural hematomas is disclosed, comprising, in combination, the steps of: inserting a dual lumen catheter into a subdural space, draining the subdural space of a subdural fluid collection with the dual lumen catheter, and irrigating the subdural space using the dual lumen catheter.

In accordance with yet another embodiment of the present invention, a dual lumen catheter is disclosed, the dual lumen catheter comprising, in combination, means for drainage and means for irrigation.

### BRIEF DESCRIPTION OF THE DRAWINGS

FIG. 1A is a side, cross-sectional view of a typical subdural space.

FIG. 1B is a side, cross-sectional view of a typical subdural hematoma.

FIG. 1C is a side, cross-sectional view of a known technique for treating subdural hematomas using multiple drill holes, known as burr-holes.

FIG. 1D is a side, cross-sectional view of a known technique for treating subdural hematomas using percutaneous catheter drainage.

FIG. 2 is a side, cross-sectional view of a technique for treating subdural hematomas using a tuohy needle with a guide wire in accordance with one embodiment of the present invention.

FIG. 3 is a side, cross-sectional view of the technique of FIG. 2, showing the dual lumen catheter guided along the guide wire in accordance with one embodiment of the present invention.

FIG. 4 is a side, cross-sectional view of the technique of FIG. 3, showing the dual lumen catheter in the subdural space after the guide wire has been removed in accordance with one embodiment of the present invention.

FIG. 5 illustrates a detailed view of the dual lumen catheter of the present invention with an irrigation container and a drainage container in accordance with one embodiment of the present invention.

FIG. 6A illustrates a detailed view of the dual lumen catheter of the present invention having a drainage channel and an irrigation channel coupled together lengthwise in accordance with one embodiment of the present invention.

FIG. 6B illustrates a detailed view of the dual lumen catheter of the present invention having an irrigation channel disposed inside the drainage channel in accordance with one embodiment of the present invention.

FIG. 7A is a side, cross-sectional view of a technique for treating subdural hematomas using a stylette with a dual lumen catheter in accordance with one embodiment of the present invention.

FIG. 7B is a side, cross-sectional view of the technique of FIG. 7A, showing the dual lumen catheter in the subdural space in accordance with one embodiment of the present invention.

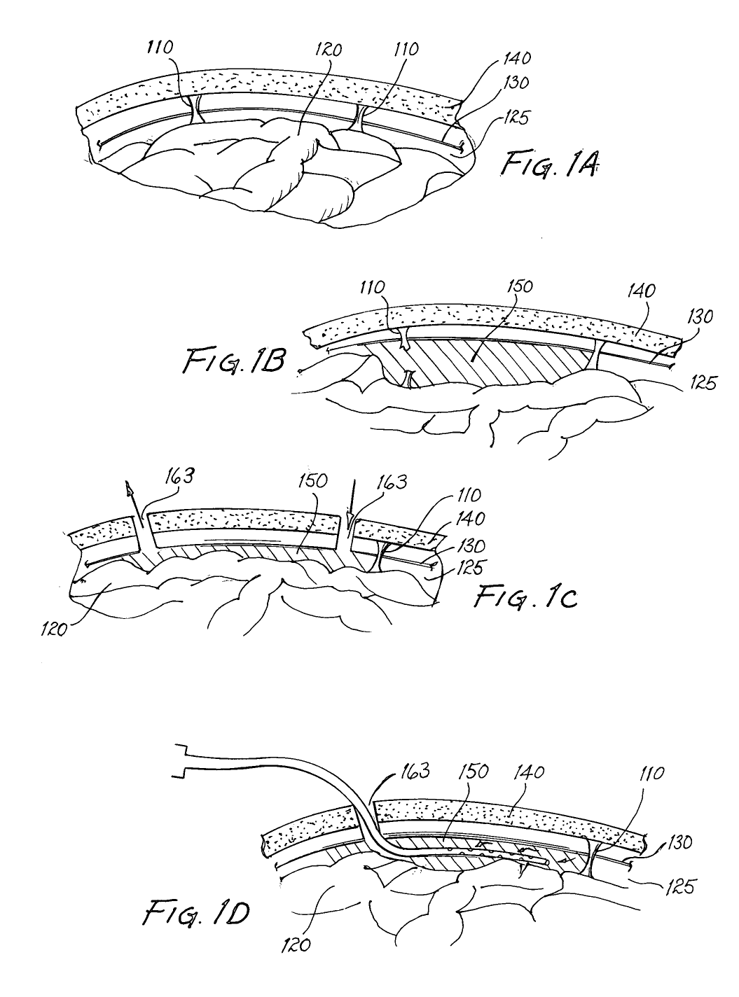

The figures depict embodiments of the present invention for purposes of illustration only. One skilled in the art will readily recognize from the following description that alternative embodiments of the structures and techniques illustrated herein may be employed without departing from the principles of the invention described herein. Common reference numerals are used throughout the drawings and detailed description to indicate like elements.

### DETAILED DESCRIPTION OF THE DISCLOSURE

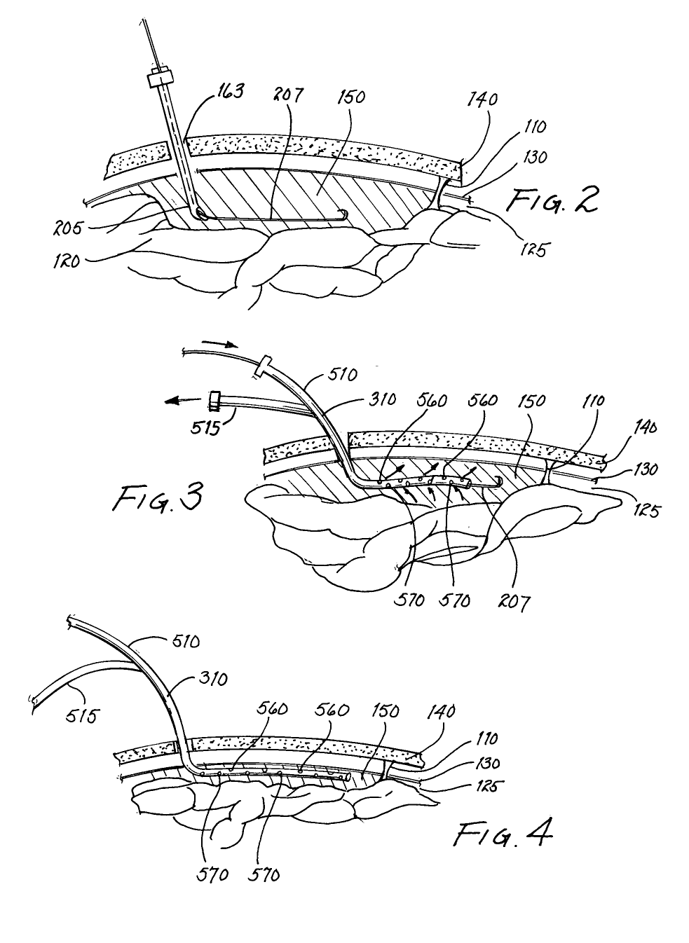

Referring to FIGS. 3-5, a dual lumen catheter, hereinafter dual lumen catheter 310, is disclosed. The dual lumen catheter 310 is for use in medical procedures for treating subdural hematomas 150. The dual lumen catheter 310 comprises a drainage channel 515 having a proximal portion 517 and a distal portion 519. The dual lumen catheter 310 also comprises an irrigation channel 510 having a proximal portion 512 and a distal portion 514. Preferably, the distal portion 519 of the drainage channel 515 is dimensioned to drain a subdural fluid collection from the brain 120 and the distal portion 512 of the irrigation channel 510 is dimensioned to irrigate the subdural space 125, although it should be clearly understood that substantial benefit could be derived from an alternative configuration of the dual lumen catheter 310 of the present invention in which the drainage channel 515 is used to drain another area of a person of an unwanted fluid collection and the irrigation channel 510 is used to irrigate the space evacuated by the unwanted fluid collection.

In the preferred embodiment, the dual lumen catheter 310 has a length of between approximately 10-30 centimeters, with the distal portion 519 of the drainage channel 515 and the distal portion 514 of the irrigation channel 510 having a length of between approximately 5-10 centimeters. Preferably, the dual lumen catheter 310 has a total diameter of between approximately 1-4 millimeters, preferably approximately 2 millimeters. It should be clearly understood, however, that substantial benefit could be derived from an alternative configuration of the dual lumen catheter 310 in which the length and diameter deviate, even substantially, from the preferred lengths and diameters in either direction.

Still referring to FIGS. 3-5, preferably the distal portion 519 of the drainage channel 515 defines drainage perforations 570 dimensioned to allow the subdural fluid collection to drain out of the subdural space 125 and through the drainage channel 515 of the dual lumen catheter 310. Preferably, each of the drainage perforations 570 are between approximately 0.5 mm and 2 mm in diameter, although it should be clearly understood that substantial benefit could be derived from an alternative configuration of the dual lumen catheter 310 in which there are no drainage perforations 570, or in which the diamter of the drainage perforations 570 deviate, even substantially, from the preferred diameter in either direction, so long as the drainage channel 515 is capable of draining a subdural fluid collection such as a subdural hematoma 150 from the subdural space 125. Preferably, the distal portion 514 of the irrigation channel 510 defines irrigation perforations 560 dimensioned to allow an irrigant to flow out of the irrigation perforations 560 and irrigate the subdural space 125. While, in the preferred embodiment, the irrigation channel 510 defines irrigation perforations 560 it should be clearly understood that substantial benefit could be derived from an alternative configuration of the irrigation channel 510 in which there is a single perforation or in which there are no irrigation perforations 560, for example if a distal end of the irrigation channel is the sole source of irrigation, so long as the irrigation channel 510 is capable of irrigating a subdural space 125.

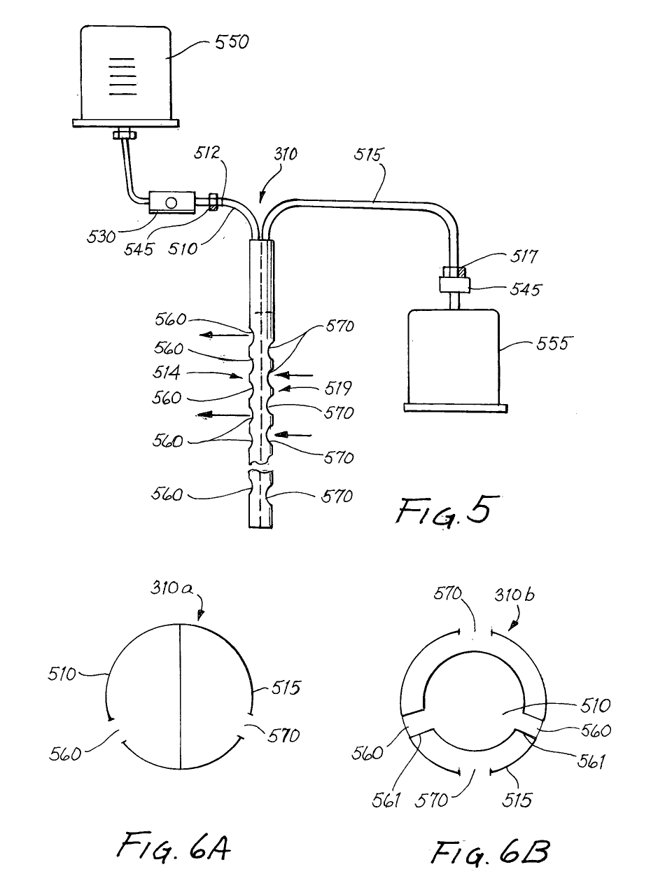

Referring specifically to FIG. 5, preferably the dual lumen catheter 310 further comprises a pressure valve 530. The pressure valve 530 is coupled, preferably with a leur lock fitting 545, to the proximal portion 512 of the irrigation channel 510. The pressure valve 530 is dimensioned to regulate a flow of fluid irrigation to the subdural space 125. Preferably, the dual lumen catheter 310 further comprises an irrigation container 550 dimensioned to retain an irrigation solution. The irrigation container 550 is preferably coupled to the pressure valve 530, although it should be clearly understood that substantial benefit could be derived from an alternative configuration of the dual lumen catheter 310 in which there is no pressure valve 530 or in which the flow of irrigation is controlled or delivered by some other means.

Still referring to FIG. 5, the dual lumen catheter 310 preferably further comprises a drainage container 555 coupled to a proximal end of the proximal portion 517 of the drainage channel 515. The drainage container 555 is dimensioned to receive subdural fluid collection from the drainage channel 515.

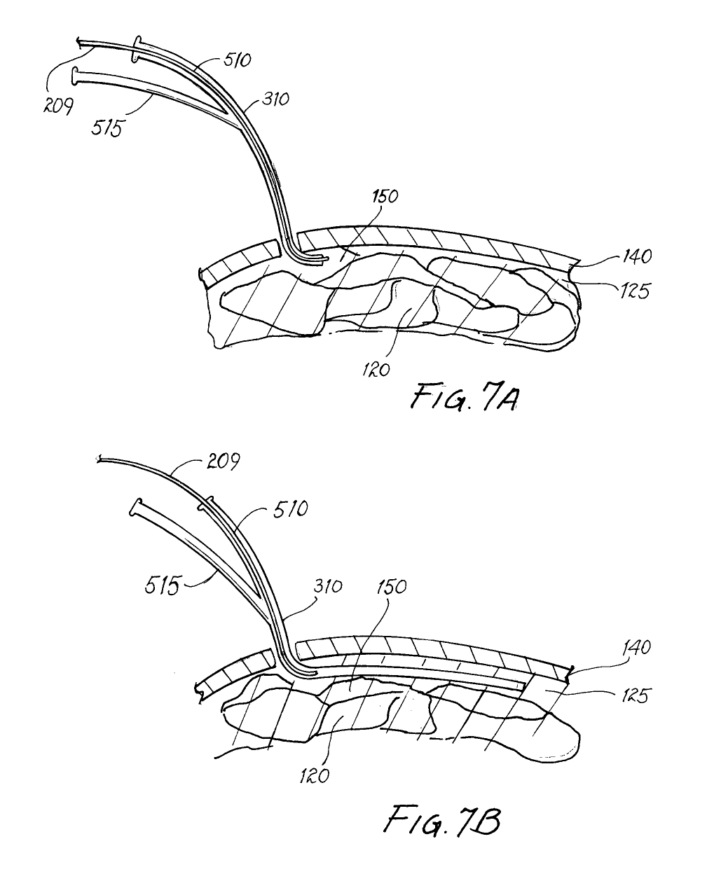

Referring now to FIGS. 6A-6B, reference numbers 310a and 310b refer to two embodiments of the dual lumen catheter (referred to generically as the dual lumen catheter 310). Referring specifically to FIG. 6A, the drainage channel 515 of the dual lumen catheter 310a is coupled lengthwise to the fluid irrigation channel 510, so that the distal portion of both the drainage channel 515 and the fluid irrigation channel 510 are side-by-side with the drainage perforations 570 preferably facing away from the irrigation channel 510 and the irrigation perforations 560 preferably facing away from the drainage channel 515.

Referring specifically to FIG. 6B, the alternative embodiment of the dual lumen catheter 310b is disclosed. In this embodiment, the irrigation channel 510 is preferably disposed inside the drainage channel 515. The irrigation channel 510 comprises a plurality of tubes 561, each having one end coupled in fluid communication to the distal portion 514 of the irrigation channel with an opposite end coupled to the drainage channel 515 so that the plurality of tubes 561 support the irrigation channel 510 inside the drainage channel 515 while at the same time the tubes 561 are dimensioned to deliver an irrigant from the irrigation channel 510 to a subdural space 125. While, in the preferred embodiment of the dual lumen catheter 310b, the irrigation channel 510 is disposed inside the drainage channel 515 it should be clearly understood that substantial benefit could be derived from an alternative configuration of the dual lumen catheter 310b in which the drainage channel 515 is disposed inside the irrigation channel 510.

Referring now to FIGS. 2-4, a technique is disclosed for treating subdural hematomas 150 using a tuohy needle 205 in accordance with one embodiment of the present invention. A tuohy needle 205 (shown in FIG. 2) has a curved tip in order to prevent penetration of the brain 120. As shown in FIG. 2, the first step of the method is for a burr hole 163 to be drilled into the skull. The tuohy needle 205 is then inserted into the subdural space 125 proximate the subdural hematoma 150 through the burr hole 163 in the skull 140. Preferably, at this point a flexible wire, known as a guide wire 207 (shown in FIGS. 2-3), is then advanced through the tuohy needle 205 parallel to the brain 120 into the subdural space 125. The tuohy needle 205 is then removed from the subdural space 125. Referring now to FIG. 3, the dual lumen catheter 310 (shown in FIGS. 3-4) is then advanced along the guide wire 207 into the subdural space 125. The guide wire 207 is then removed from the subdural space 125 (as shown in FIG. 4). The dual lumen catheter 310, appropriately positioned in the area of the subdural hematoma 150, can then begin to drain and irrigate in order to effectively evacuate the subdural hematoma 150. Referring specifically to FIG. 4, the subdural space 125 is thereby effectively collapsed, and the brain returns to be adjacent to the dura matter 130 and skull 140. In this way, not only is the subdural fluid collection evacuated, but the subdural space 125 is thoroughly irrigated in order to cleanse the subdural space of subdural fluid collection residue. While, in the preferred method, a tuohy needle 205 and a guide wire 207 are used, it should be clearly understood that substantial benefit could be derived from an alternative method of treatment in which the tuohy needle 205 is used without a guide wire 207, or in which neither the tuohy needle 205 or the guide wire 207 are used. For example, the dual lumen catheter 310 may be inserted directly into the subdural space 125 through a burr hole 163.

As is known in the art, the tuohy needle 205 and flexible wire 207 can be selected from appropriate commercially available devices for use in medical procedures. Examples of appropriate commercially available devices are available from Arrow (Central Venous Catheterization Kit) and Beckton Dickenson (Tuohy needle).

Referring now to FIGS. 7A-7B an alternative method for treating subdural hematomas 150 is disclosed. In this method, a stylette 209 is used. A stylette 209 is a thin wire or strip of metal capable of being bent but also retaining its shape and rigidity. Instead of using a tuohy needle 205, the stylette 209 is inserted directly into the dual lumen catheter 310 in order to give the dual lumen catheter 310 rigidity. The stylette 209 is preferably bent in a substantially L-shaped configuration in order to give the dual lumen catheter 310 this shape. The dual lumen cathether 310 is then inserted into the subdural space 125 and guided over the stylette 209 parallel to the brain 120. The stylette 209 is then removed from the dual lumen catheter 310. The dual lumen catheter 310, appropriately positioned in the area of the subdural hematoma 150, can then begin to drain and irrigate in order to effectively evacuate the subdural hematoma 150. While, in the preferred method, a stylette 209 is used, it should be clearly understood that substantial benefit could be derived from an alternative method of treatment in which a stylette 209 is not used so long as the dual lumen catheter 310 can effectively be inserted into the subdural space 125 in order to evacuate the subdural hematoma 150 and irrigate the subdural space 125.

In the preferred embodiment, the method of percutaneous drainage utilizing the dual lumen catheter 310 occurs over a period of approximately three days, with the irrigation occurring for approximately one to two days therein. While, in the preferred embodiment, the dual lumen catheter 310 is preferably utilized at a patient's bedside over the course of three days, it should be clearly understood that substantial benefit could be derived from an alternative use of the dual lumen catheter in which the duration of usage deviates, even substantially, from the preferred usage in either direction. For example, in addition to bedside use, it is within the spirit and scope of this invention that the dual lumen catheter 310 may be used in an operating room environment for a much shorter period of time, and then either removed or left in for an extended duration of drainage and irrigation.

Accordingly, the present invention allows for an improved technique to perform treatment of subdural hematomas. The present invention does not require the drilling of multiple burr holes in the skull but yet also effectively washes out coagulation products. The above description is included to illustrate the operation of the preferred embodiments and is not meant to limit the scope of the invention. From the above discussion, many variations will be apparent to one skilled in the relevant art that would yet be encompassed by the spirit and scope of the invention.

1. An apparatus for use in medical procedures for treating subdural hematomas, the apparatus comprising a dual lumen catheter comprising, in combination:
a drainage channel having a proximal portion and a distal portion; and
an irrigation channel having a proximal portion and a distal portion.

2. The apparatus of claim 1 wherein said drainage channel defines drainage perforations proximate said distal portion of said drainage channel and said irrigation channel defines irrigation perforations proximate said distal portion of said drainage channel.

3. The apparatus of claim 2 wherein each of said drainage perforations having a diameter of between approximately 0.5 and 2 millimeters.

4. The apparatus of claim 1 further comprising a pressure valve coupled to said proximal portion of said irrigation channel, said pressure valve dimensioned to regulate a flow of fluid irrigation.

5. The apparatus of claim 4 further comprising an irrigation container dimensioned to retain an irrigation solution, said container being coupled to said pressure valve.

6. The apparatus of claim 5 further comprising a leur lock fitting connecting said container to said pressure valve.

7. The apparatus of claim 1 further comprising a drainage container coupled to a proximal end of said proximal portion of said drainage channel, said drainage container dimensioned to receive subdural fluid collection from said drainage channel.

8. The apparatus of claim 1 wherein said dual lumen catheter having a length of between approximately 10 and 30 centimeters.

9. The apparatus of claim 1 wherein said distal portion of said drainage channel being coupled lengthwise to said distal portion of said fluid irrigation channel.

10. The apparatus of claim 1 wherein said distal portion of said drainage channel and said distal portion of said fluid irrigation channel have a length of between approximately 5 and 10 centimeters.

11. The apparatus of claim 1 wherein said irrigation channel being disposed inside said drainage channel, said irrigation channel comprising a plurality of tubes each having one end coupled in fluid communication to said distal portion of said irrigation channel, each opposite end of said plurality of tubes coupled to said drainage channel so that said plurality of tubes support said irrigation channel inside said drainage channel while at the same time said plurality of tubes being dimensioned to deliver an irrigant from said irrigation channel to a subdural space.

12. The apparatus of claim 1 wherein said dual lumen catheter having a diameter of between approximately 1 and 4 millimeters.

13. The apparatus of claim 12 wherein said dual lumen catheter having a diameter of approximately 2 millimeters.

14. A method for treating subdural hematomas comprising, in combination, the steps of:
inserting a dual lumen catheter into a subdural space;
draining said subdural space of a subdural fluid collection with said dual lumen catheter; and
irrigating said subdural space using said dual lumen catheter.

15. The method of claim 14 further comprising the steps of:
providing a drainage channel having a proximal portion and a distal portion;
providing an irrigation channel having a proximal portion and a distal portion;
wherein said drainage channel and said irrigation channel comprise said dual lumen catheter;
draining said subdural space of a subdural fluid collection using said drainage channel of said dual lumen catheter; and
irrigating said subdural space using said irrigation channel of said dual lumen catheter.

16. The method of claim 15 further comprising the steps of:
draining said subdural space of subdural collection fluid through perforations defined by said drainage channel; and
irrigating said subdural space through perforations defined by said irrigation channel while draining of said subdural space by said drainage channel is performed.

17. The method of claim 16 wherein each of said drainage perforations having a diameter of between approximately 0.5 and 2 millimeters.

18. The method of claim 15 further comprising the steps of:
providing a pressure valve coupled to said proximal portion of said irrigation channel; and
operating said pressure valve in order to regulate a flow of fluid irrigation from said pressure valve to said irrigation channel.

19. The method of claim 18 further comprising the steps of:
providing an irrigation container dimensioned to retain an irrigation solution;
coupling said container to said pressure valve; and
operating said pressure valve in order to regulate a flow of fluid irrigation from said pressure valve to said irrigation channel.

20. The method of claim 19 further comprising the step of coupling said container to said pressure valve with a leur lock fitting.

21. The method of claim 15 further comprising the steps of:
providing a drainage container dimensioned to receive subdural collection fluid from said drainage channel;
coupling said drainage container to a proximal end of said proximal portion of said drainage channel; and
draining said subdural space of said subdural collection fluid so that said drainage container fills with said subdural collection fluid from said subdural space.

22. The method of claim 14 further comprising the step of drilling a hole into a skull.

23. The method of claim 14 further comprising the steps of:
providing a tuohy needle;
drilling a hole in a skull;
inserting said tuohy needle into said subdural space of said skull;
inserting said dual lumen catheter into said tuohy needle; and
removing said tuohy needle from said subdural space.

24. The method of claim 14 further comprising the steps of:
providing a tuohy needle;
drilling a hole in a skull;
inserting said tuohy needle into said subdural space of said skull;
inserting a guide wire into said tuohy needle approximately parallel to the brain;
removing said tuohy needle from said subdural space;
advancing said dual lumen catheter along said guide wire into said subdural space; and
removing said guide wire from said subdural space.

25. The method of claim 14 further comprising the steps of:
drilling a hole in a skull;
inserting a stylette into said dual lumen catheter in order to give said dual lumen catheter rigidity;
inserting said dual lumen catheter into said subdural space; and
removing said stylette from said dual lumen catheter.

26. The method of claim 14 wherein said draining of said subdural space occurring over approximately three days.

27. The method of claim 14 wherein said irrigating of said subdural space occurring over approximately between 1-2 days.

28. A dual lumen catheter for treatment of subdural hematomas, the dual lumen catheter comprising, in combaintion:
means for drainage; and
means for irrigation.

29. The apparatus of claim 28 wherein the means for drainage further comprises a drainage container coupled to the means for drainage.

30. The apparatus of claim 28 wherein the dual lumen catheter further comprises:
perforation means for drainage; and
perforation means for fluid irrigation.

31. The apparatus of claim 28 wherein the dual lumen catheter further comprises a pressure regulating means for regulating fluid pressure.

32. The apparatus of claim 28 wherein the dual lumen catheter further comprises:
a first lock means for connecting an irrigation container for irrigating a subdural hematoma; and
a second lock means for connecting a drainage container for draining said subdural hematoma.

33. The apparatus of claim 28, further comprising a means for inserting the dual lumen catheter into a subdural space.
